(19) United States
(12) Patent Application Publication
(10) Pub. No.: US 2007/0053486 A1
(43) Pub. Date: Mar. 8, 2007

(54) Methods and apparatus for nuclear tomo-cardiology scanning

(76) Inventors: Deborah Ruth Zelnik, Haifa (IL); Pascal Salazar-Ferrer, Eden Prairie, MN (US)

Correspondence Address: THE SMALL PATENT LAW GROUP LLP, 611 OLIVE STREET, SUITE 1611, ST. LOUIS, MO, 63101, US

(21) Appl. No.: 11/490,611

(22) Filed: Jul. 21, 2006

Related U.S. Application Data
(60) Provisional application No. 60/710,436, filed on Aug. 23, 2005.

(51) Int. Cl. A61B 6/00; G01N 23/00; G21K 1/12; H05G 1/60

(52) U.S. Cl. 378/20

(57) ABSTRACT

Methods and systems for medical imaging are provided. The system includes a medical imaging system with a scanner having a gantry and a camera aligned with a center of rotation axis of the gantry. The medical imaging system further includes a display configured to display an image of the gantry from the camera.

### CROSS REFERENCE TO RELATED APPLICATIONS

This application claims priority to and the benefit of the filing date of U.S. Provisional Application No. 60/710,436, filed on Aug. 23, 2005, entitled “METHODS AND APPARATUS FOR NUCLEAR TOMO-CARDIOLOGY IMAGING,” which is hereby incorporated by reference in its entirety.

### BACKGROUND OF THE INVENTION

This invention relates generally to medical imaging systems and, more particularly, to patient positioning in a medical imaging system.

When imaging using nuclear tomo-cardiology scanning, patient positioning is typically important for proper imaging. For example, imaging is facilitated and can be improved when a patient is positioned such that the heart of the patient is axially aligned with the Center of Rotation (COR) of the gamma camera detectors of the scanner, such as centered within a bore of the scanner. If a patient is off-center, the image may be difficult to review/evaluate and may not provide the necessary image information to perform a proper diagnosis.

In some known systems, alignment is achieved by manually adjusting the table height until the patient's torso is centered within the COR of the detectors. This manual action often requires bending, leaning, etc. on the part of the operator in order to place the operator's eye in a direct horizontal line with the COR and to eyeball alignment of the patient within the scanner. This process can lead to less than optimal imaging conditions because it may be difficult for an operator to accurately determine when a patient is properly aligned within the scanner. Accordingly, less than optimal images may result. Additionally, multiple manual adjustments may be needed, thereby adding time to the scanning process. Further, the scanner may be positioned such that viewing positioning adjustments may be almost impossible, for example, if the detector side of the scanner is close to a wall.

Additionally, default positions are defined in some gamma camera systems. However, even with the defined default positions, because of differences in patient size, scanner configurations, etc., manual adjustment is often still needed to accommodate each patient and align the patient within the scanner.

### BRIEF DESCRIPTION OF THE INVENTION

In one embodiment, a medical imaging system is provided that includes a scanner having a gantry and a camera aligned with a center of rotation axis of the gantry. The medical imaging system further includes a display configured to display an image of the gantry from the camera.

In another embodiment, a medical imaging system display is provided that includes an image of a gantry of a medical imaging scanner and an indication of a center of rotation axis of the medical imaging scanner.

In yet another embodiment, a method for providing alignment information for a medical imaging system is provided. The method includes acquiring an image of a center of rotation axis of a medical imaging scanner and displaying the image on a display of the medical imaging system.

### BRIEF DESCRIPTION OF THE DRAWINGS

FIG. 1 is a perspective view of an exemplary imaging system.

FIG. 2 is a schematic block diagram of the imaging system shown in FIG. 1.

FIG. 3 is a perspective view of a nuclear tomo-cardiology scanner constructed in accordance with an exemplary embodiment of the invention.

FIG. 4 is another perspective view of the nuclear tomo-cardiology scanner shown in FIG. 3 including a camera workstation and display monitor.

FIG. 5 is a display of the display monitor of FIG. 4 illustrating an exemplary image of a gantry bore and patient table as viewed through the video camera shown in FIG. 3.

FIG. 6 is a display of the display monitor of FIG. 4 illustrating another exemplary image of a gantry bore and patient table as viewed through the video camera shown in FIG. 3.

### DETAILED DESCRIPTION OF THE INVENTION

As used herein, an element or step recited in the singular and proceeded with the word “a” or “an” should be understood as not excluding plural elements or steps, unless such exclusion is explicitly recited. Furthermore, references to “one embodiment” of the present invention are not intended to be interpreted as excluding the existence of additional embodiments that also incorporate the recited features.

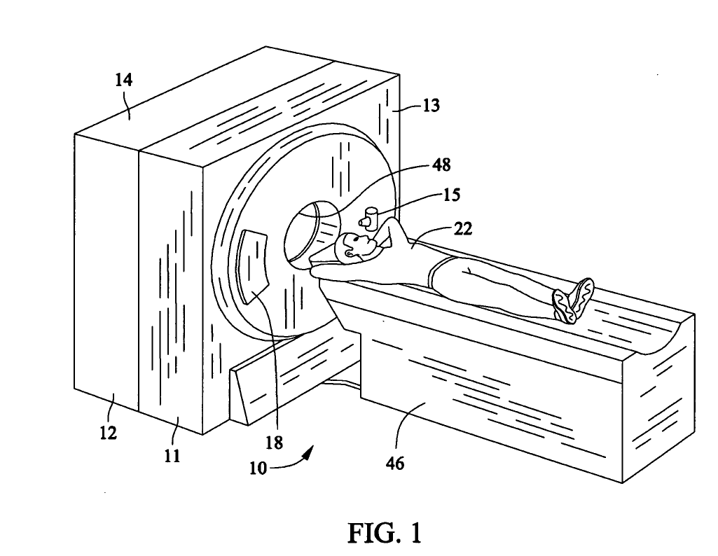

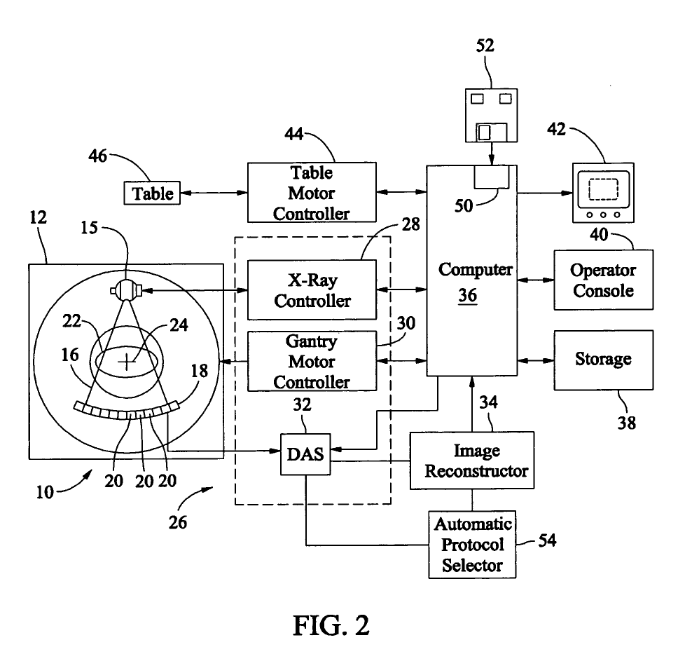

FIG. 1 is a perspective view of an exemplary imaging system 10 in connection with which various embodiments of the invention may be implemented and operated. FIG. 2 is a schematic block diagram of the imaging system 10 (shown in FIG. 1). Referring now to FIGS. 1 and 2, in an exemplary embodiment, the imaging system 10 is a multi-modal imaging system and includes a first modality unit 11 and a second modality unit 12. The modality units 11 and 12 enable the system 10 to scan an object, for example, a patient, in a first modality using the first the modality unit 11 and to scan the object in a second modality using the second modality unit 12. The system 10 allows multiple scans in different modalities. In one embodiment, the multi-modal imaging system 10 is a Computed Tomography/Positron Emission Tomography (CT/PET) imaging system 10. The CT/PET system 10 includes a first gantry 13 associated with the first modality unit 11 and a second gantry 14 associated with the second modality unit 12. In alternative embodiments, modalities other than CT and PET may be employed with the imaging system 10, for example, x-ray, magnetic resonance (MR), etc. In the embodiment shown, the gantry 13 includes the first modality unit 11 that has an x-ray source 15 that projects a beam of x-rays 16 toward a detector array 18 on the opposite side of the gantry 13. The detector array 18 is formed by a plurality of detector rows (not shown) including a plurality of detector elements 20 that together sense the projected x-rays that pass through an object, such as a patient 22. Each detector element 20 produces an electrical signal that represents the intensity of an impinging x-ray beam and allows estimation of the attenuation of the beam as the beam passes through an object, such as the patient 22.

In other embodiments, the system 10 includes only a single gantry having a first rotor configured to carry the first modality and a second rotor configured to carry the second modality. In various other embodiments, the system 10 includes only a single gantry that performs imaging in one modality, such as CT, MR, PET or x-ray, among others.

During a scan to acquire imaging data, for example, x-ray projection data, the gantry 13 and the components mounted thereon rotate about an examination axis 24. FIG. 2 shows only a single row of detector elements 20 configured as a single detector row. However, the detector array 18 may be configured as a multislice detector array having a plurality of parallel detector rows of detector elements 20 such that projection data corresponding to a plurality of slices can be acquired simultaneously during a scan. Additional imaging data also may be acquired, for example, if multiple modalities are used. For example, to acquire emission data, the gantry 14 rotates one or more gamma cameras (not shown) about the examination axis 24. The gantry 14 may be configured for continuous rotation during an imaging scan and/or for intermittent rotation between imaging frames.

The rotation of the gantries 13 and 14, and the operation of the x-ray source 15 are controlled by a control unit 26 of the imaging system 10. The control unit 26 includes controllers, for example, an x-ray controller 28 that provides power and timing signals to the x-ray source 15 and a gantry motor controller 30 that controls the rotational speed and position of the gantry 13 and the gantry 14. A data acquisition system (DAS) 32 of the control unit 26 samples data from the detector elements 20 and the gamma cameras and conditions the data for subsequent processing. In the exemplary embodiment, an image reconstructor 34 receives sampled and digitized x-ray data and emission data from the DAS 32 and performs image reconstruction. The reconstructed image is transmitted as an input to a computer 36 that stores the image in a storage device 38.

The computer 36 also receives commands and scanning parameters from an operator via a console 40 that has an input device, such as, a keyboard, mouse, roller ball, etc. An associated display 42 allows the operator to observe the reconstructed image and other data from the computer 36 as described in more detail herein. The operator supplied commands and parameters are used by the computer 36 to provide control signals and information to the DAS 32, the x-ray controller 28 and the gantry motor controller 30. In addition, the computer 36 operates a table motor controller 44 that controls a motorized table 46 to position the patient 22 in the gantry 13 and 14. Specifically, the table 46 moves portions of the patient 22 through a gantry opening 48 and is configured to align the patient therein, for example, using forward/backward and upward/downward movement.

In one embodiment, the computer 36 includes a read/write device 50, for example, a floppy disk drive, CD-ROM drive, DVD drive, magnetic optical disk (MOD) device, or any other digital device including a network connecting device such as an Ethernet device for reading instructions and/or data from a computer-readable medium 52, such as a floppy disk, a CD-ROM, a DVD or an other digital source such as a network or the Internet, as well as yet to be developed digital means. In another embodiment, the computer 36 executes instructions stored in firmware (not shown). The computer 36 is programmed to perform functions as described herein, and as used herein, the term computer is not limited to integrated circuits referred to in the art as computers, but broadly refers to computers, processors, microcontrollers, microcomputers, programmable logic controllers, application specific integrated circuits, and other programmable circuits, and these terms are used interchangeably herein. The imaging system 10 may include a particular type of detector depending on the imaging modality. For example, a plurality of PET detectors (not shown) including a plurality of detector elements may be provided. The PET detectors and the detector array 18 both detect radiation and are both referred to herein as radiation detectors.

An automatic protocol selector 54 is communicatively coupled to the DAS 32 and the image reconstructor 34 to transmit settings and parameters for use by the DAS 32 and the image reconstructor 34 during a scan and/or image reconstruction and image review. Although the automatic protocol selector 54 is illustrated as a separate component, it should be understood that that functions performed by the automatic protocol selector 54 may be incorporated into functions performed by, for example the computer 36. Accordingly, the automatic protocol selector 54 may be embodied in a software code segment executing on a multifunctional processor or may embodied in a combination of hardware and software.

Additionally, although described in a medical setting, it is contemplated that the embodiments of the invention may be implemented in connection with other imaging systems including, for example, industrial CT systems such as, for example, but not limited to, a baggage scanning CT system typically used in a transportation center, such as, an airport or a rail station.

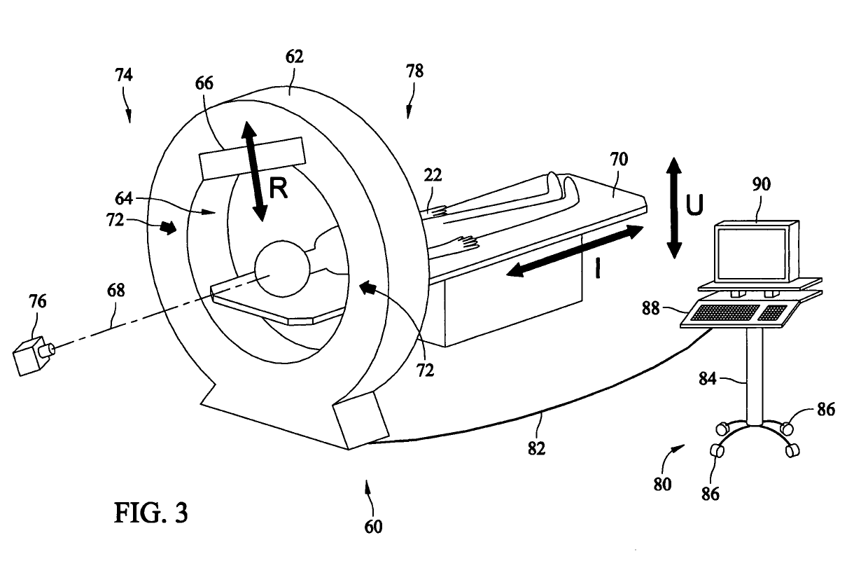

FIG. 3 is a perspective view of a nuclear tomo-cardiology scanner 60 constructed in accordance with an embodiment of the invention and that may be used with the imaging system 10 (shown in FIGS. 1 and 2). However, the scanner 60 also may be used with other types of medical and non-medical imaging systems. The scanner 60 includes a gantry 62 that is substantially cylindrical and includes an opening 64 therethrough defining a gantry bore. The gantry 62 is configured to support at least one imaging device or detector 66, for example, a nuclear gamma camera and to rotate the detector 66 about a Center of Rotation (COR) axis 68. The detector 66 also may be configured for radial movement, for example, radial inward and outward movement relative to the gantry 62 and illustrated by the arrow “R”. A patient table 70, which may be the same or similar to the table 46 (shown in FIG. 1) is provided and configured to move the patient 22 supported thereon in to and out of the gantry 62, and in particular, in to and out of the opening 62 as illustrated by the arrow “I”. The patient table 70 also is configured to move upward and downward as illustrated by the arrow “U” to position the patient 22 within the opening as described in more detail herein.

The gantry 62 also includes one or more markings 72 on one side of the gantry 62, for example, a camera side 74 of the gantry 62. The markings 72 may be configured as index marks that are vertically aligned with the COR axis 68. A camera, for example, a video camera 76 is positioned on the camera side 74 of the gantry 62, which is generally opposite a patient table side 78 of the gantry 62. The video camera 76 is oriented such that the viewing axis of the video camera 76 is aligned with the COR axis 68. The video camera 76 may be any type of video camera 76 and may be mounted or supported in any manner, for example, on a tripod or mounted to a wall.

A scanner workstation 80 also may be provided and connected to the scanner 60. The connection may be provided via a wired link 82, for example, a wired cable. Alternatively, communication between the scanner workstation 80 and the scanner 60 may be provided via a wireless link. The scanner workstation 80 generally includes a stand 84, that may be movable (e.g., on wheels 86) and that supports a user input, such as a keyboard 88 and a display, such as, a monitor 90 (e.g., an acquisition console screen). The workstation 80 also may include a processing unit and communication unit (both not shown) provided in any known manner.

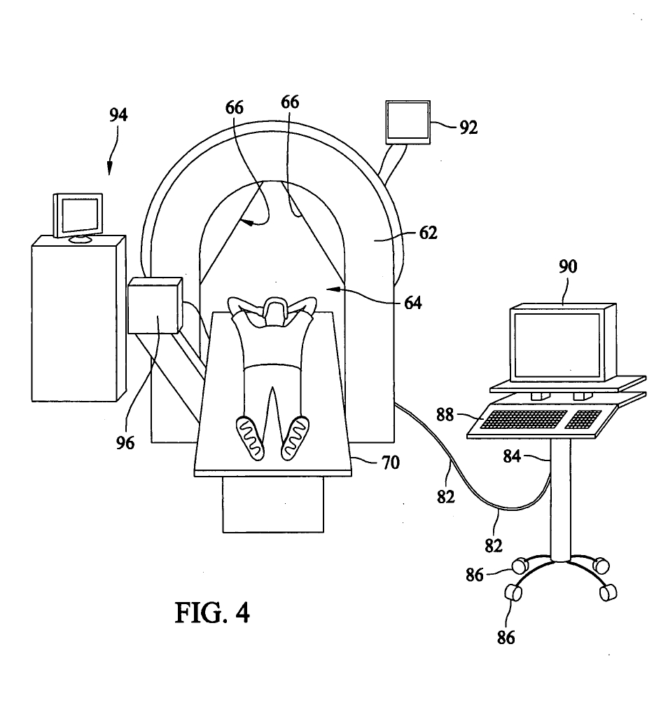

Additional or different components also may be provided, for example, a display 92 as shown in FIG. 4. The display 92 may be mounted to a wall or supported on a stand and continuously display the patient 22 or the gantry 62. It should be noted that the display 92 may be provided in addition to a display (e.g., monitor) of the workstation 80 or instead of the display of the workstation 80 as a dedicated display. Further, additional computing units 94 for processing and viewing acquired data may be provided. Also, other monitoring equipment may be provided in connection with the scanner 60, for example, an electrocardiogram (ECG) unit 96 mounted to the side of the scanner 60.

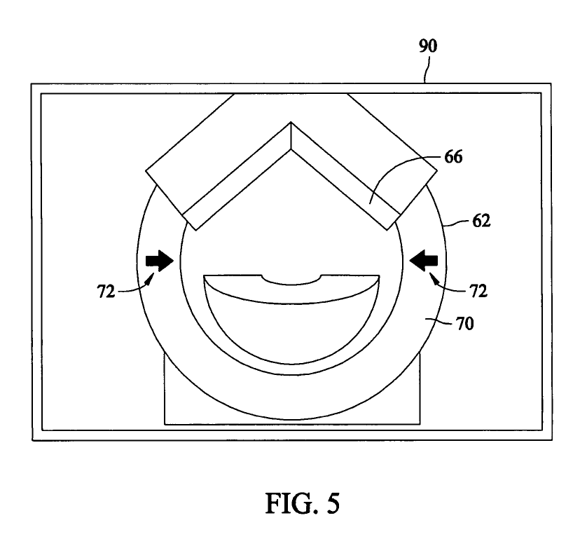

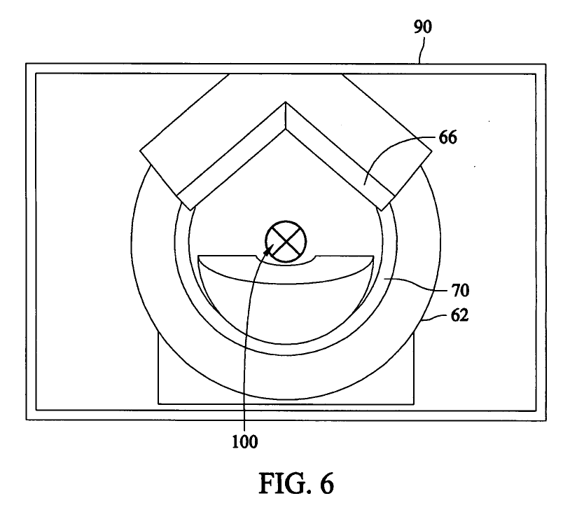

Referring to the workstation 80, the monitor 90 displays the gantry 60 as shown in FIG. 5 such that an operator can view the relative position of the patient table 70, and more particularly, the patient 22 (shown in FIGS. 3 and 4), to the markings 72. It should be noted that the image displayed on the monitor 90 also may be displayed on the display 92. In another embodiment, as shown in FIG. 6, an overlay 100 may be provided on the monitor 90 indicating the COR axis 68 shown in FIG. 3. The overlay 100 may be configured, for example, as a circle or oval with crosshairs to indicate the COR axis 68 of the gantry 62.

In operation, and referring to FIGS. 3 through 6, the positioning and alignment of the patient table 70 is controlled from the scanner workstation 80, which controls movement of the patient table 70 in at least one of an axial direction and a vertical or radial direction. Movement of the patient table 70 may be controlled with user commands provided at the user input 88 and that controls operation of the table motor controller 44 (shown in FIG. 2). For example, a user may control movement of the patient table 70 to substantially align the patient 22 with the markings 72 or the overlay 100 or both. As an example for use in tomo-cardiology scanning, during operation, the patient 22 is positioned on the patient table 70. The position of the patient table 70 is then controlled using the workstation 80 such that the heart of the patient 22 is substantially aligned with the COR axis 68. In the exemplary embodiment, the video camera 76 receives an image of the opening 64 defining the bore of the gantry 62 with the patient 22 in approximate alignment with the COR axis 68. This approximate alignment may be provided by a default centering position for the scanner 60 or by eyeball alignment by an operator. The image is transmitted to the workstation 80 (e.g., via wired or wireless communication) where the image is displayed on the monitor 90. The operator uses the image of the gantry 62 and the patient 22 to position the patient 22 (e.g., fine adjustments) such that, for example, the heart of the patient 22 is substantially aligned with the COR axis 68 of the gantry 62 as determined by the markings 72 and/or the overlay 100.

In another exemplary embodiment, software executing on the workstation 80 controls the patient table 70 to provide automatic alignment. For example, the software receives the image of the gantry 62 and the patient 22, determines an outline of the periphery of the patient 22 using, for example, any known edge detection program, determines an axis of the patient 22 that includes the patient heart area (e.g., based on the typical position of a patient heart), and generates patient table positioning control commands that automatically substantially align the patient heart area with the COR axis 68. This may include an iterative process wherein the alignment of the patient 22 is compared multiple times to the COR axis 68 to align the patient 22 within a predetermined variance of the COR axis 68. The detection of the various areas within the image may be provided with any known graphics processing program. This automatic alignment process may include, for example, the imaging software first receiving a reference image of the gantry 62 and patient table 70 without the patient 22 in position on the patient table 70. For each new scan, the user commands the video camera 76 to transmit an image of the patient 22 on the patient table 70. The software then compares the reference image to the current patient image to determine the outline of the periphery of the patient 22. The software then determines a heart area of the patient 22 using predetermined geometries of the periphery of the patient 22 and or learned geometries. The markings 72 located on the gantry 62 and/or the overlay 100 facilitate aligning the heart area of the patient 22 with the COR axis 68 of the gantry 62. Manual adjustments also may be made as needed or desired.

The software also may be selectively configured to apply virtual reference marks on the image to facilitate alignment, for example, aligning the patient heart area with the COR axis 68. For example, the software may be configured to outline the patient periphery with selectively colored lines, outline the determined patient heart area, and provide the overlay 100, for example, configured as targeting crosshairs over the COR axis 68 of the gantry 62 and the patient heart area.

The various embodiments also may provide additional functionality or information in connection with the displayed image. For example, an operator may be able to select regions of the image with a corresponding description displayed. Additionally, the display may provide indications (e.g., arrows) that provide a proposed realignment motion. For example, using quantitative feedback, the direction and size of the indications may provide guidance as to the direction and amount of movement needed to align the patient 22 or the portion of interest of the patient 22 with the COR axis 68. The image also may be modified, for example, moved, enlarged, etc. or may be provided in connection with other scanning information.

It should also be noted that additional visual aids may be provided in connection with the gantry 62 for use when aligning the patient 22. For example, instead of or in addition to the markings 72, indentations may be provided on the material that forms a portion of, for example, a head support for the patient.

Thus, various embodiments of the invention provide an image of a gantry along a COR that may be displayed on one or more displays including, for example, a monitor of a workstation and/or additional monitors within an examination room. A technical effect of the various embodiments of the systems and methods described herein include facilitating alignment of a patient within a gantry using displayed images and visual indications. The visual indications may be provided physically or virtually. For example, the above-described embodiments of a medical imaging system provide a cost-effective and reliable means for automatically aligning a patient with a gantry center of rotation in a reproducible orientation during a nuclear tomo-cardiology scan.

Although various embodiments are described above relative to a nuclear medicine system, other medical imaging modalities, such as computed tomography (CT), single positron emission tomography (SPECT), positron emission tomography (PET), nuclear magnetic resonance imaging (MRI), static X-ray imaging, dynamic (Fluoroscopy) X-ray imaging, and multimodality combinations thereof, among others, may also benefit from the methods described herein and the use of the various embodiments of the present invention is contemplated with respect to these modalities

Exemplary embodiments of medical imaging systems and apparatus are described above in detail. The medical imaging system components illustrated are not limited to the specific embodiments described herein, but rather, components of each system may be utilized independently and separately from other components described herein. For example, the medical imaging system components described above may also be used in combination with different medical imaging system components.

While the invention has been described in terms of various specific embodiments, those skilled in the art will recognize that the invention can be practiced with modification within the spirit and scope of the claims.

What is claimed is:

1. A medical imaging system comprising:
a scanner including a gantry;
a camera aligned with a center of rotation axis of the gantry; and
a display configured to display an image of the gantry from the camera.

2. A medical imaging system in accordance with claim 1 wherein the camera comprises a video camera oriented with a viewing axis of the video camera aligned with the center of rotation axis.

3. A medical imaging system in accordance with claim 1 wherein the image is configured to provide patient positioning information.

4. A medical imaging system in accordance with claim 1 wherein the display further comprises a virtual indication of the center of rotation axis.

5. A medical imaging system in accordance with claim 4 wherein the virtual indication comprises crosshairs.

6. A medical imaging system in accordance with claim 1 wherein the gantry further comprises markings indicating the center of rotation axis.

7. A medical imaging system in accordance with claim 1 further comprising a patient head support comprising indentations indicating the center of rotation axis.

8. A medical imaging system in accordance with claim 1 further comprising a patient table configured to be manually aligned within the gantry based on the displayed image.

9. A medical imaging system in accordance with claim 1 further comprising a patient table configured to be automatically aligned within the gantry based on the displayed image.

10. A medical imaging system in accordance with claim 1 further comprising a patient table and wherein the camera is provided on an opposite side of the gantry to the patient table.

11. A medical imaging system in accordance with claim 1 wherein the display is provided on at least one of a wall, a stand and as part of a workstation.

12. A medical imaging system in accordance with claim 1 wherein the scanner comprises a nuclear medicine scanner.

13. A medical imaging system in accordance with claim 1 wherein the image is communicated to the display via a wireless link.

14. A medical imaging system display comprising:
an image of a gantry of a medical imaging scanner; and
an indication of a center of rotation axis of the medical imaging scanner.

15. A medical imaging system display in accordance with claim 14 wherein the indication comprises a virtual overlay.

16. A medical imaging system display in accordance with claim 14 wherein the indication comprises a physical marking on the gantry.

17. A method for providing alignment information for a medical imaging system, the method comprising:
acquiring an image of a center of rotation axis of a medical imaging scanner; and
displaying the image on a display of the medical imaging system.

18. A method in accordance with claim 17 further comprising displaying a virtual indication of the center of rotation axis on the display.

19. A method in accordance with claim 17 further comprising displaying a physical indication of the center of rotation axis on the display.

20. A method in accordance with claim 17 further comprising aligning a patient within a gantry of a scanner of the medical imaging system using the displayed image.
